(19) United States
(12) Patent Application Publication
(10) Pub. No.: US 2016/0216191 A1
(43) Pub. Date: Jul. 28, 2016

(54) Methods For Predicting Asphaltene Precipitation

(71) Applicant: BP Corporation North America Inc., Naperville, IL (US)

(72) Inventors: Soban Balashanmugam, Surrey (GB); Mehdi Haghshenas, Houston, TX (US); Doris Gonzalez, Katy, TX (US); Timothy Totton, Surrey (GB)

(73) Assignee: BP Corporation North America Inc., Naperville, IL (US)

(21) Appl. No.: 14/943,517

(22) Filed: Nov. 17, 2015

Related U.S. Application Data
(60) Provisional application No. 62/106,326, filed on Jan. 22, 2015.

(51) Int. Cl. G01N 13/00; G01N 33/28

(52) U.S. Cl. CPC G01N 13/00; G01N 33/2811

(57) ABSTRACT

A method predicts the asphaltene precipitation envelope of a fluid consisting of stock tank oil and dissolved gas. The method comprises comparing a solubility parameter of the fluid, δ_{fluid}, and an onset solubility parameter of the fluid, δ_{onset(fluid)}, across a range of pressures, to predict pressures at which asphaltene precipitation will be observed, δ_{fluid} and δ_{onset(fluid)} are calculating using a correction factor, F_{correction}. F_{correction} is determined according to formula (1):

F_{correction}=δ_{STO(physical)}/δ_{STO(solvent power)} (1)
where: δ_{STO(physical)} is an estimate of the solubility parameter of the stock tank oil based on a physical property of the stock tank oil, and
δ_{STO(solvent power)} is an estimate of the solubility parameter of the stock tank oil based on the solvent power of the stock tank oil.

### FIELD OF THE INVENTION

The present invention relates to methods for predicting the asphaltene precipitation envelope and related parameters. In particular, the present invention relates to methods for predicting the asphaltene precipitation envelope and related parameters of a fluid from a subterranean formation.

### BACKGROUND OF THE INVENTION

Hydrocarbon fluid production requires complex subsea and surface production systems which are designed to safely extract hydrocarbons from a hydrocarbon fluid producing reservoir. The fluid is typically extracted under extreme pressure and temperature conditions, particularly when it is being extracted from deepwater reservoirs.

The fluid which is extracted typically contains hydrocarbon solids such as wax, hydrates and asphaltenes. The deposition of these hydrocarbon solids in the production system can create significant disruption to overall operations. For instance, asphaltenes can deposit in any one or all of the well-bore, the manifold, flowlines/risers and topsides.

Asphaltene deposition is largely a composition and pressure driven phenomenon, with temperature playing a secondary role. In particular, under-saturated, high pressure reservoirs with a high gas to hydrocarbon fluid ratio tend to exhibit the highest risk of asphaltene deposition.

Under-saturated hydrocarbon fluid reservoirs are not fully saturated with dissolved gas. As the pressure of the hydrocarbon fluid is reduced on extraction, the gas remains in solution until the oil bubble point of the fluid is reached. As the pressure rises from reservoir pressure to the oil bubble point, dissolved gas components in the hydrocarbon fluid start to expand, resulting in a decrease in the fluid density and increased molar volume of the fluid.

The increasing molar volume results in a reduction in the solvent power (SP) and the solubility parameter (δ) of the hydrocarbon fluid. These are simple metrics used to define the ability of a fluid to dissolve asphaltenes. A higher solvent power and solubility parameter gives better dissolution of asphaltenes.

When the solvent power falls below the asphaltene critical solvent power of the hydrocarbon fluid (CSPa), or the solubility parameter falls below the onset solubility parameter of the hydrocarbon fluid (δonset), asphaltenes from the hydrocarbon fluid begin to precipitate. Increasing quantities of asphaltenes will precipitate out from the fluid with a greater difference between the solvent power and the asphaltene critical solvent power, or the solubility parameter and the onset solubility parameter.

Accordingly, it can be seen that the presence of dissolved gas in a fluid can act as an asphaltene precipitant.

The upper asphaltene onset pressure is the pressure above the oil bubble point at which asphaltenes start to precipitate from the hydrocarbon fluid. The lower asphaltene onset pressure is the pressure below the oil bubble point at which asphaltenes stop precipitating from the hydrocarbon fluid. As the pressure falls during hydrocarbon fluid extraction, asphaltene precipitation starts at the upper asphaltene onset pressure and occurs until the lower asphaltene onset pressure is reached.

It will be appreciated that methods which predict the asphaltene precipitation envelope may be very useful when it comes to designing or operating hydrocarbon fluid extraction systems. The asphaltene precipitation envelope may be used to assess the asphaltene deposition risk. For instance, knowledge of the asphaltene precipitation envelope enables locations to be identified which may be prone to asphaltene instability and thus help devise suitable mitigation and/or remediation strategies.

The ASIST (ASphaltene InStability Trend) method is widely known, and is based on the fundamental assumption that the solubility parameter and the refractive index of non-polar substances such as crude oils are linearly related. However, predictions of the asphaltene onset pressures that are made using the ASIST method generally do not match with the measured asphaltene onset pressures of fluids. The ASIST method is described by Wang et al.: An Experimental Approach to Prediction of Asphaltene Flocculation (SPE 64994, 2001).

Asphaltene onset pressure can also be measured on live fluids by depressurization experiments performed on live fluids in a Solids Detection System (SDS) apparatus. However, this process is expensive and laborious, requiring both specialist equipment and live downhole fluid samples.

Accordingly, there is a need for a method which reliably predicts the asphaltene precipitation envelope of a fluid.

### SUMMARY OF THE INVENTION

The present invention provides a method for determining a solubility parameter of a stock tank oil, δ_{STO}, at one or more pressures, said method comprising:

determining a correction factor, F_{correction}, according to formula (1):

F_{correction}=δ_{STO(physical)}/δ_{STO(solvent power)} (1)

wherein: δ_{STO(physical)} is an estimate of the solubility parameter of the stock tank oil based on a physical property of the stock tank oil, and
δ_{STO(solvent power)} is an estimate of the solubility parameter of the stock tank oil based on the solvent power of the stock tank oil,

applying the correction factor, F_{correction}, to an estimate of the solubility parameter of the stock tank oil based on a physical property of the stock tank oil, δ_{STO(estimated)}, at one or more pressures, according to formula (2):

δ_{STO}=δ_{STO(estimated)}/F_{correction} (2).

The present invention further provides a method for estimating a solubility parameter of a fluid consisting of stock tank oil and dissolved gas, δ_{fluid}, at one or more pressures, said method comprising calculating δ_{fluid} according to formula (3):

δ_{fluid}=V_{(fraction DG)}*δ_{DG}+V_{(fraction STO)}*δ_{STO} (3)

wherein: V_{(fracton DG)} is a volume fraction of the dissolved gas,
δ_{DG} is a solubility parameter of the dissolved gas,
V_{(fraction STO)} is a volume fraction of the stock tank oil, and
δ_{STO} is a solubility parameter of the stock tank oil,

wherein δ_{STO} is determined according to a method as defined herein.

The present invention also provides a method for predicting an onset solubility parameter of a fluid consisting of stock tank oil and dissolved gas, δ_{onset(fluid)}, at one or more pressures, said method comprising:

titrating the stock tank oil against two or more titrants to determine, for each titrant, a volume fraction of the stock tank oil at the onset of asphaltene precipitation, V_{(onset fraction STO)}, a volume fraction of the titrant at the onset of asphaltene precipitation, V_{(onset fraction T)}, and a root molar volume of precipitants at the onset of asphaltene precipitation, vₚ^{0.5}_{(STO+T)};

calculating an onset solubility parameter of the stock tank oil with each titrant, δ_{onset(STO+T)}, according to formula (4):

δ_{onset(STO+T)}=V_{(onset fraction T)}*δ_{T}+V_{(onset fraction STO)}*δSTO (4)

wherein: δ_{T} is a solubility parameter of the titrant, and
δ_{STO} is a solubility parameter of the stock tank oil;

determining a relationship between δ_{onset(STO+T)} and vₚ^{0.5}_{(STO+T)}; and

predicting δ_{onset(fluid)} from a root molar volume of dissolved gas in the fluid, vₚ^{0.5}_{(fluid)}, based on the relationship between δ_{onset(STO+T)} and vₚ^{0.5}_{(STO+T)},

wherein δ_{STO} is determined according to a method as defined herein.

The present invention further provides a method for predicting an asphaltene precipitation envelope of a fluid consisting of stock tank oil and dissolved gas, said method comprising comparing a solubility parameter of the fluid, δ_{fluid}, and an onset solubility parameter of the fluid, δ_{onset(fluid)}, across a range of pressures, to predict pressures at which asphaltene precipitation will be observed, wherein:

a solubility parameter of the stock tank oil, δ_{STO}, is used to determine δ_{fluid} and δ_{onset(fluid)} across the range of pressures and is determined according to a method as defined herein.

Also provided is a method for mitigating the deposition of asphaltenes from a fluid consisting of a stock tank oil and dissolved gas in a fluid extraction process, said method comprising predicting the asphaltene precipitation envelope of the fluid using a method as defined herein, and modifying the fluid extraction process so that the deposition of asphaltenes is reduced.

### BRIEF DESCRIPTION OF THE DRAWINGS

FIG. 1 depicts the asphaltene precipitation envelope (10) for a fluid;

FIG. 2 depicts a graph of δonset(STO+T) against vp0.5(STO+T) for a fluid from the Gulf of Mexico (fluid A);

FIG. 3 depicts a graph of fluid and Sonset(fluid) across the range of pressures measured for fluid A; and

FIG. 4 depicts a graph comparing the direct measurement with the predicted measurement of the onset volumes of a series of commingled stock tank oils with each titrant.

### DETAILED DESCRIPTION OF THE INVENTION

By applying the correction factor, F_{correction}, during the methods of the present invention, an improved prediction of the solubility parameter of the stock tank oil may be obtained which, in turn, leads to an improved predictions of the asphaltene precipitation envelope, the solubility parameter of a fluid and the onset solubility parameter of a fluid. In particular, the asphaltene precipitation envelope may be predicted with good agreement with the measured asphaltene precipitation envelope for a fluid. The methods of the present invention represent an improvement on known methods, such as the ASIST method described above.

Moreover, the methods of the present invention enable accurate prediction of the asphaltene precipitation envelope, and related parameters, from just a PVT report (i.e. Pressure-Volume-Temperature data) and a small sample of stock tank oil.

In some instances, the method for predicting the asphaltene precipitation envelope of a fluid comprises carrying out the abovementioned steps of the method for predicting the solubility parameter of a fluid, δ_{fluid}. In some instances, the method for predicting the asphaltene precipitation envelope of a fluid comprises carrying out the abovementioned steps of the method for predicting the onset solubility parameter of a fluid, δ_{onset(fluid)}. In some instances, the method for predicting the asphaltene precipitation envelope of a fluid comprises carryout the abovementioned steps of the method for predicting the solubility parameter of a fluid, δ_{fluid}, and the abovementioned steps of the method for predicting the onset solubility parameter of a fluid, δ_{onset(fluid)}.

If, at a particular pressure, δ_{fluid} is lower than δ_{onset(fluid)}, then asphaltene precipitation is predicted to occur. If, at a particular pressure, δ_{onset(fluid)} is lower then δ_{fluid}, then asphaltene precipitation is not predicted to occur. A graph of δ_{fluid} and δ_{onset(fluid)} across the range of pressures measured may be plotted so that the asphaltene precipitation envelope (if present) may be visualized. The upper asphaltene onset pressure and the lower asphaltene onset pressure may be estimated, for instance from the graph.

In some instances, δ_{fluid}, δ_{onset(fluid)} and δ_{STO} are determined over a range of pressures. For instance, δ_{fluid}, δ_{onset(fluid)} and δ_{STO} may be determined at two or more pressures, such as at 5 or more pressures, or at 10 or more pressures. The pressures may be in the range of from 2,000-140,000 kPa, such as from 3,500-45,000 kPa.

In some instances, δ_{fluid}, δ_{onset(fluid)} and δ_{STO} are determined at reservoir temperature. For instance, δ_{fluid}, δ_{onset(fluid)} and δ_{STO} may be determined at a temperature in the range of from 30-200° C., such as from 80-130° C. In instances, δ_{fluid}, δ_{onset(fluid)} and δ_{STO} may be determined across a range of temperatures, for instance at two or more temperatures, such as 5 or more temperatures. The temperatures may be in the range of from 30-200° C.

The correction factor, F_{correction}

In order to calculate the correction factor, F_{correction}, it is necessary to determine δ_{STO(physical)}, an estimate of the solubility parameter based on physical parameters of the stock tank oil, and δ_{STO(solvent power)}, a solubility parameter based on the solvent power of the stock tank oil.

δ_{STO(physical)} is an estimate of the solubilityparameter of the stock tank oil based on a physical property of the stock tank oil. Suitable physical properties include the density of the stock tank oil and the refractive index of the stock tank oil.

In some instances, δ_{STO(physical)} may be calculated according to formula (5):

δ_{STO(physical)}=52.042*(RI_{STO}²−1)/(RI_{STO}²+2)+2.904 (5)

where: RI_{STO} is the refractive index of the stock tank oil.

The refractive index of the stock tank oil, RI_{STO}, may be measured experimentally using known methods. For instance, RI_{STO} may be measured according to ASTM D 1747-09. RI_{STO} may be measured at temperatures falling within the range of from 15-90° C., such as from 20-60° C., and at atmospheric pressure, i.e. 100 kPa.

In other instances, δ_{STO(physical)} may be calculated according to formula (6):

δ_{STO(physical)}=17.347*ρ_{STO}+2.904 (6)

where: ρ_{STO} is the density of the stock tank oil.

The density of the stock tank oil, ρ_{STO}, may be measured experimentally using known methods. For instance, ρ_{STO} may be measured according to ASTM D 4052 or D 5002. ρ_{STO} will typically be measured at room temperature and atmospheric pressure, i.e. 20° C. and 100 kPa, though it may be measured at temperatures of up to 200° C. and pressures of up to 140,000 kPa using a high pressure-high temperature densitometer, such as an Anton-Paar device.

It will be appreciated that formulae (5) and (6) are essentially equivalent, since it is known that the density of hydrocarbons is typically three times (RI_{STO}²=1)/(RI_{STO}²+2). Further explanation in this regard can be found in Zuo, J. Y. et al: A Simple Relation between Solubility Parameters and Densities for Live Reservoir Fluids (J. Chem. Eng. Data, 55 (2010) 2964-2969) and Vargas, F. M. et al: Application of the One-Third rule in hydrocarbon and crude oil systems (Fluid Phase Equilibria, 290 (2010) 103-108), the disclosures of which are incorporated herein by reference.

δ_{STO(solvent power)} is an estimate of the solubility parameter based on the solvent power of the stock tank oil. Any known method may be used to determine the solvent power of the stock tank oil. For instance, the methodology described in Patent US 2004/0121472 (Nemana, S. et al: Predictive Crude Oil Compatibility Model; incorporated herein by reference) may be used, according to which oil solvent power is estimated using the Watson K factor.

The Watson K factor, K_{STO}, is calculated according to formula (7):

K_{STO}=VABP_{STO}^{1/3}/SG_{STO} (7)

where: VABPsro is the volume average boiling point of the stock tank oil, in degrees

### Rankine, and

SG_{STO} is the standard specific gravity of the stock tank oil.

The volume average boiling point of the stock tank oil, VABP_{STO}, may be determined using known methods. In some instances, VABP_{STO} may be determined from the yield profile of the stock tank oil.

The yield profile of the stock tank oil may be determined from physical distillation, for instance according to ASTM D 2892 or ASTM D 5236. The yield profile of the stock tank oil may alternatively be determined using GC and high temperature simulated distillation (HT-SIMDIS). Use of GC analysis allows the hydrocarbon composition of the oil to be determined for components boiling in the C₁₋₉ hydrocarbon range. GC analysis may be carried according to standard test method IP PM-DL. HT-SIMDIS analysis may be carried out according to standard test method IP 545.

The standard specific gravity of the stock tank oil, SG_{STO}, is the ratio of the density of the stock tank oil to that of water at 60° F. (i.e. 15.6° C.). SG_{STO} may be determined using known methods. For instance, as mentioned above, the density of the stock tank oil may be measured experimentally according to ASTM D 4052 or D 5002. The density of the stock tank oil may also be determined from the yield profile of the oil, for instance using a simulation tool (such as HYSYS) which may predict the density of the stock tank oil at 60° F.

The solvent power of the stock tank oil, SP_{STO}, may be determined from the Watson K factor using linear interpolation. For instance, SP_{STO} may be determined from K_{STO} based on the relationship between the Watson K factor and the solubility parameter of heptane and toluene. The Watson K factor and the solubility parameter of heptane and toluene are known in the art.

The solubility parameter of the stock tank oil based on the solvent power of the stock tank oil, δ_{STO(solvent power)}, may be determined from the solvent power of the stock tank oil, SP_{STO}, also using linear interpolation. For instance, δ_{STO(solvent power)} may be determined from SP_{STO} based on the relationship between the solvent powers and solubility parameters of heptane and toluene. The solvent powers and solubility parameters of heptane and toluene are known in the art.

F_{correction} is a coefficient which is assumed to be substantially independent of pressure and temperature. Accordingly, a similar value is assumed to be obtained, regardless of the pressure or temperature at which F_{correction} is determined.

F_{correction} may be determined at a single pressure. In other instances, for greater accuracy, F_{correction} may be determined at more than one pressure. F_{correction} may be obtained at a single pressure such as at atmospheric pressure, i.e. 100 kPa, or at one or more pressures up to 140,000 kPa.

Where F_{correction} is determined at one or more pressures, it is calculated as the mean average of values determined at more than one pressure. For instance, where δ_{STO(physical)} and δ_{STO(solvent power)} are determined at a first pressure to an nth pressures, F_{correction}=[δ_{STO(physical at P1)}/δ_{STO(measured at P1)}+δ_{STO(physical at P2)}/δ_{STO(measured at P2)}+ . . . δ_{STO(physical at Pn)}/δ_{STO(measured at Pn)}]/n, where P1 is the first pressure, P2 is the second pressure and Pn is the nth pressure. n is preferably from 2-5. Generally, determination of δ_{STO(physical)} and δ_{STO(solvent power)} at a single pressure provides a correction factor, F_{correction}, with sufficient accuracy for use in the methods of the present invention.

F_{correction} may be determined at a single temperature. In other instances, F_{correction} may be determined at more than one temperature. F_{correction} may be obtained at room temperature, i.e. 20° C., or at one or more temperatures up to 200° C. Where F_{correction} is the mean average of values determined at more than one temperature, it will be understood that, as with pressure, each value is determined at a single temperature.

Accordingly, the skilled person will appreciate that although the value obtained for F_{correction} is assumed to be substantially independent of pressure and temperature, when calculating F_{correction}, δ_{STO(physical)} and δ_{STO(solvent power)} should be determined at the same temperature and pressure.

Since the value obtained for F_{correction} is substantially independent of pressure and temperature, it may be applied across the range of pressures or temperatures at which δ_{fluid} and δ_{onset(fluid)} are estimated, irrespective of the one or more pressures and temperatures at which δ_{STO(physical)} and δ_{STO(solvent power)} are determined. The skilled person will appreciate that the temperature and pressure should be kept consistent for parameters described herein other than F_{correction}, i.e. only those parameters obtained at the same pressure and temperature should be combined.

### The Solubility Parameter of the Stock Tank Oil, δ_{STO}

The solubility parameter of the stock tank oil, δ_{STO}, is calculated from F_{correction}, the correction factor, and δ_{STO(estimated)}, the estimate of the solubility parameter of the stock tank oil based on a physical property of the stock tank oil. Suitable physical properties include the density of the stock tank oil and the refractive index of the stock tank oil. For instance, δ_{STO(estimated)} may be calculated according to formula (8):

δ_{STO(estimated)}=17.347*ρ_{STO}+2.904 (8)

where: ρ_{STO} is the density of the stock tank oil.

At some pressures, i.e. those close to atmospheric pressure, the density of the stock tank oil, ρ_{STO}, may simply be measured using the methods mentioned above.

However, across a wider pressure range, such as that typically encountered in reservoirs, ρ_{STO} at one or more pressures may be predicted by determining the yield profile of the stock tank oil, and using the yield profile to predict the density of the stock tank oil.

The yield profile of the stock tank oil may be analysed using GC and HT-SIMDIS. GC and HT-SIMDIS analysis may be carried according to standard test methods mentioned above, i.e. standard test methods IP PM-DL and IP 545, respectively.

A simulation tool (such as HYSYS) may be used to predict the density of the stock tank oil, ρ_{STO}, at a wide range of pressures and temperatures. Typically, the simulation tool will slice the yield profile into groups of components with similar boiling points, which then enables the prediction of the stock tank oil density at a wide range of pressures and temperatures. It will be appreciated, that by using GC, HT-SIMDIS and HYSYS, the density of the stock tank oil may be estimated, and so it does not need to be measured at high temperature and high pressure.

Alternatively, δ_{STO(estimated)} may be calculated according to formula (9):

δ_{STO(estimated)}=52.042*(TI_{STO}²−1)/(RI_{STO}²+2)+2.904 (9)

where: RI_{STO} is the refractive index of the stock tank oil.

The refractive index of the stock tank oil, RI_{STO}, may be measured experimentally using known methods. For instance, RI_{STO} may be measured as outlined above.

Since it is desirable to assess δ_{STO(estimated)} across a wide range of pressures, as found in a reservoir, then δ_{STO(estimated)} will typically calculated based on ρ_{STO}.

The Solubility_Parameter of the Fluid, δ_{fluid}

As mentioned above, the solubility parameter of the fluid, δ_{fluid}, may be calculated according to formula (3):

δ_{fluid}=V_{(fraction DG)}*δ_{DG}+V_{(fraction STO)}*δ_{STO} (3)

where:V_{(fraction DG)} is a volume fraction of the dissolved gas,
δ_{DG} is a solubility parameter of the dissolved gas,
V_{(fraction STO)} is a volume fraction of the stock tank oil, and
δ_{STO} is a solubility parameter of the stock tank oil.

The solubility parameter of the dissolved gas, δ_{DG}, may be estimated based on a physical property of the dissolved gas. Physical properties include the density of the dissolved gas. For instance, δ_{DG} may be calculated according to formula (10):

δ_{DG}=17.347*ρ_{DG}+2.904 (10)

where: ρ_{DG} is the density of the dissolved gas.

The density of the dissolved gas, ρ_{DG}, at one or more pressures may be determined from the composition of the dissolved gas in the fluid. In some instances, the dissolved gas is represented by the C₁₋₆ paraffin components of the fluid.

The composition of the dissolved gas may be determined by known methods. For instance, the composition of the dissolved gas may be derivable from PVT data, such as single stage flash data.

At pressures lower than 3,500 kPa, the composition of the dissolved gas may change, due to evaporation of the heavier components, such as the C₄₋₆ paraffin components. To determine the composition of the dissolved gas in the live fluid at pressures of less than 3,500 kPa, then a simulator tool may be used, such as MultiFlash or PVTSim.

The density of the dissolved gas, ρ_{DG}, at different pressures may be determined from the composition of the dissolved gas using equations of state, such as the Peng-Robinson or Soave-Redlich-Kwong equations of state.

Alternatively, the density of the dissolved gas may be determined by direct measurement of the fluid, e.g. at temperatures up to 200° C. and pressures up to 140,000 kPa using a high pressure-high temperature densitometer, such as an Anton-Paar device. However, it is preferred that the density of the dissolved gas be determined from the PVT data.

There is no need to apply a correction factor when detenuining the solubility parameter of the dissolved gas, δ_{DG}.

Methods for measuring the solubility parameter of the stock tank oil, δ_{STO} are given above.

The volume fraction of the dissolved gas, V_{(fraction DG)}, and the volume fraction of the stock tank oil, V_{(fraction STO),} may be measured using any known method. In some instances, V_{(fraction DG}) will be determined, and V_{(fraction STO)} will be determined according to the relationship V_{(fraction STO)}=1−V_{(fraction DG)}.

In some instances, V_{(fraction DC)} and V_{(fraction STO)} may be derived from PVT data on the fluid. In particular, V_{(fraction DG)} and V_{(fraction STO)} may be derived at one or more pressures from the differential liberation residual oil density, the gas to oil ratio, the density of the stock tank oil, ρ_{STO}, and the density of the dissolved gas, ρ_{DG}. Methods for measuring the density of the stock tank oil and the dissolved gas are provided above.

In these instances, V_{(fraction STO)} is calculated assuming that the overall “shrinkage” of the mixture when the dissolved gas and stock tank oil are combined is fully absorbed by the gas phase. This is a reasonable assumption given that the mass of dissolved gas in the live fluid is significantly lower than that of the stock tank oil.

The Onset Solubility Parameter of the Fluid, δ_{onset(fluid)}

As mentioned above, the onset solubility parameter of the fluid, δ_{onset(fluid)}, may be predicted, at one or more pressures, by titrating the stock tank oil against two or more titrants.

The titrants may be two or more different n-paraffins. In some instances, at least three different n-paraffins are used. In some instances, the titrants are selected from heptane, undecane and pentadecane.

The period of time for which the stock tank oil and the titrant are equilibrated may be from 20-40 minutes, such as 30 minutes. These equilibration times improve the quality of the data which is obtained, due to minimized heating times and improved test turnaround times. In some instances, the stock tank oil and the titrant are undisturbed during this time, i.e. they are not subjected to any mixing or agitation. Aliquots of stock tank oil and titrant may be prepared so that the precipitation onset volume may be determined to a precision of at least 5% by volume, such as at least 2% by volume. The stock tank oil and titrant mixtures may be observed under an optical microscope to determine when asphaltene precipitation occurs.

Accordingly, it can be seen that the volume fraction of the stock tank oil at the onset of asphaltene precipitation, V_{(onset fraction STO)}, and a volume fraction of the titrant at the onset of asphaltene precipitation, V_{(onset fraction T)} are determined from the titrations. In some instances, V_{(onset fraction T)} will be measured, and V_{(onset fraction STO)} will be determined based on the relationship as V_{(onset fraction STO)}=1−V_{(onset fraction T)}.

The root partial molar volume of precipitants at the onset of asphaltene precipitation, vₚ^{0.5}_{(STO+T)}, may be determined using known methods. For instance, vₚ^{0.5}_{(STO+T)} may be determined using a simulation tool, such as HYSYS, and equations of state, such as the Peng-Robinson equations of state.

The onset solubility parameter of the stock tank oil with each titrant, δ_{onset(STO+T)}, is calculated according to formula (4):

δ_{onset(STO+T)}=V_{(onset fraction T)}*δ_{T}+V_{(onset fraction STO)}*δSTO (4).

The solubility parameter of the titrant, δ_{T}, may be determined at one or more pressures experimentally, or may be known in the art. Where δ_{T} is determined experimentally, it may be determined based on the density or the refractive index of the titrant. For instance, δ_{T} may be calculated according to formula (11):

δ_{T}=17.347*ρ_{T}+2.904 (11)

where: ρ_{T} is the density of the titrant.

Densities of titrant are known in the art, or may be determined using standard methods.

Alternatively, δ_{T} may be calculated according to formula (12):

δ_{T}=52.042*(RI_{T}²−1)/(RI_{T}²+2)+2.904 (12)

where: RI_{T} is the refractive index of the titrant.

The refractive index of the titrant, RI_{T}, may be known in the art, or may be determined experimentally using standard methods.

δ_{STO} is the solubility parameter of the stock tank oil and is determined as described above, using F_{correction}.

In some instances, the method for predicting the onset solubility parameter of the fluid, δ_{onset(fluid)}, is carried out at a temperature which is close to that of the reservoir temperature.

However, in most instances, it will be necessary to modify the method so that the findings can be extrapolated to reservoir temperature. In these instances, the method involves titrating the stock tank oil against two or more titrants at two or more temperatures with each titrant. In other words, at least four separate titrations are performed (two titrants, at two temperatures each).

The test temperatures should be above the Wax Appearance Temperature (WAT) of the titrant. Typically, titrations may be carried out with each titrant at three temperatures. In some instances, the temperatures are selected from 40, 50 and 60° C.

Determination of δ_{onset(STO+T)} and vₚ^{0.5}_{(STO+T)} at two or more temperatures enables, by extrapolation, δ_{onset(STO+T)} and vₚ^{0.5}_{(STO+T)} to be determined at reservoir temperature. The relationships between δ_{onset(STO+T)} and temperature, and between δ_{onset(STO+T)} and vₚ^{0.5} _{(STO+T)}, are assumed to be linear. As mentioned above, reservoir temperature typically falls within the range of from 30-200° C., such as from 80-130° C.

Once δ_{onset(STO+T)} and vₚ^{0.5}_{(STO+T)} are known for two or more titrants, for instance at reservoir temperature, a relationship between δ_{onset(STO+T)} and vₚ^{0.5}_{(STO+T)} may be determined. As mentioned, the relationship is assumed to be a linear relationship. In some instances, it may be desirable to plot a graph of 67 _{onset(STO+T)} against vₚ^{0.5}_{(STO+T)}, though the relationship can also be determined without the need to plot a graph.

The onset solubility parameter of the fluid, δ_{onset(fluid)}, may then be predicted from the root partial molar volume of dissolved gas in the fluid, vₚ^{0.5}_{(fluid)}, based on the relationship between δ_{onset(STO+T)} and vₚ^{0.5}_{(STO+T)}. This is because the relationship between δ_{onset(STO+T)} and vₚ^{0.5}_{(STO+T)} is assumed to be the same as the relationship between δ_{onset(fluid)} and vₚ^{0.5}_{(fluid)}.

The root partial molar volume of dissolved gas in the fluid, vₚ^{0.5}_{(fluid)}, may be derived from PVT data on the fluid. In particular, vₚ^{0.5}_{(fluid)} may be derived at one or more pressures from the differential liberation residual oil density, the gas to oil ratio and the density of the stock tank oil, ρ_{STO}.

### The Fluid

The fluid referred to herein is typically a downhole fluid, such as a hydrocarbon fluid which is present in a subterranean formation (commonly referred to as a live fluid). The fluid will typically be extracted from the subterranean formation as crude oil.

The fluid consists of stock tank oil and dissolved gas. Accordingly, removal of the dissolved gas from the fluid gives oil which is considered, for the purposes of the present invention, to be stock tank oil. Stock tank oil may be obtained by bringing the fluid to atmospheric conditions, for instance of 20° C. and 100 kPa.

The stock tank oil is preferably free from any asphaltene inhibitors. The stock tank oil is preferably free from any dispersants. The stock tank oil is preferably free from drilling mud, and any other contaminants.

Typically, 400 cm³ of stock tank oil will be suitable for carrying out the analysis required by the method of the present invention. The stock tank oil may be obtained from surface separators, or from down-hole fluid that has been depressurized and returned to ambient pressure.

### Comingled Systems

In some instances, the method of the present invention is used to predict the asphaltene precipitation envelope of a single fluid. In other instances, the fluid may be a comingled fluid which is formed from two or more separate fluids. Comingled fluids are common where an oil reservoir has multiple wells producing from different “sands”. The properties, e.g. composition, density, asphaltene content and gas to oil ratio, of fluids from each producing sand may be very different, and asphaltene precipitation may vary between separate fluids. The mixing of two or more separate fluid streams may serve to increase, decrease or have no impact on the asphaltene precipitation for the commingled system.

Commingled fluids may be assessed by carrying out the methods outlined above on the comingled fluid, for instance by using PVT data for the comingled fluid (or predicting it using a tool such as PVTSim) and a stock tank oil sample from the comingled fluid.

In other instances, comingled fluids may be assessed by carrying out the methods outlined above on the separate fluids that combine to make the comingled fluid. The pressure and temperature at which the comingling occurs may be readily determined from operating data.

As before, the correction factor, F_{correction}, for a comingled fluid may be determined from δ_{STO(physical)} and δ_{STO(solvent power)}. However, with a comingled fluid, δ_{STO(physical)} and δ_{STO(solvent power)} are determined by % blending, such as volume % blending, for each of the separate fluids which form the comingled fluid.

Accordingly, where the comingled fluid is formed from n separate fluids, δ_{STO(physical)}=[δ_{STO(physical of F1)}*volume % of F1+δ_{STO(physical of F2)}*volume % of F2+ . . . δ_{STO(physical of Fn)*volume % of Fn], where F}1 is the first fluid, F2 is the second fluid and Fn is the nth fluid which forms the comingled fluid. Similarly, δ_{STO(solvent power)}=[δ_{STO(solvent power of F1)}*volume % of F1+67 _{STO(solvent power of F2)}*volume % of F2+ . . . δ_{STO(solvent power of Fn)}*volume % of Fn], where F1 is the first fluid, F2 is the second fluid and Fn is the nth fluid which forms the comingled fluid.

It will be appreciated that the volume % blending may be carried out at any appropriate stage during the calculation of F_{correction}. For instance, in the case of δ_{STO(solvent power)}, % blending calculations may be carried out in order to determine the solvent power of the comingled fluid, from which Ogro (solvent power) may be determined directly without further % blending considerations.

The volume % blending of the separate fluids which form the comingled fluid may be determined using known methods. For instance, the volume % blending may be readily determined from operating data.

As before, the solubility parameter of the stock tank oil, δ_{STO}, for a comingled fluid is calculated from F_{correction}, the correction factor, and κ_{STO(estimated)}, which may be calculated based on the density of the stock tank oil, ρ_{STO}, for the comingled fluid.

ρSTO may be determined for the comingled fluid using known methods. In some instances, ρ_{STO} may be determined by determining the yield profile for each of the separate fluids which form the comingled fluid, and using a blend assay tool (such as CrudeSuite). The density of the comingled fluid can be predicted at a wide range of pressures and temperatures using a tool such as HYSYS.

As before, the solubility parameter of the comingled fluid, δ_{fluid}, may be calculated from V_{(fraction DG)}, δ_{DG}, V_{(fraction STO)}, and δ_{STO}.

δDG may be estimated based on the density of the dissolved gas, ρ_{DG}, in the comingled fluid. This may be determined % blending, such as volume % blending, the composition of the dissolved gas in each of the separate fluids which form the comingled fluid. The density of the dissolved gas, ρ_{DG}, in the comingled fluid at one or more different pressures may then be determined using equations of state.

V_{(fraction DG)} and V_{(fraction STO)} for the comingled fluid may be derived from the PVT data on each of the separate fluids which form the comingled fluid. An equations of state tool, such as PVTSim, may be used to determined V_{(fraction DG)} and V_{(fraction STO)} for the comingled fluid.

Methods for determining δ_{STO} for a comingled fluid are discussed above.

As above, the onset solubility parameter of the comingled fluid, δ_{onset(fluid)}, may be predicted from a root partial molar volume of dissolved gas in the comingled fluid, vₚ^{0.5}_{(fluid)}, based on the relationship between δ_{onset(STO+T)} with vₚ^{0.5}_{(STO+T)}.

The root partial molar volume of dissolved gas in the fluid, vₚ^{0.5}_{(fluid)}, may be derived from PVT data on the separate fluids which form the comingled fluid.

vₚ^{0.5}_{(STO+T)} for the comingled fluid is simply a function of the titrants used during the experiment and do not vary when a commingled fluid is used.

As above, the onset solubility parameter of the comingled stock tank oil with each titrant, δ_{onset(STO+T)}, may be calculated from V_{(onset fraction T)}, δ_{T}, V_{onset fraction STO)} and δ_{STO}.

Methods for determining δ_{STO} for a comingled stock tank oil are discussed above.

Methods for determining δ_{T} are discussed above, and do not vary when a comingled stock tank oil is used.

V_{(onset fraction STO)} may be determined for the comingled stock tank oil from V_{(onset fraction T)} for the comingled stock tank oil. Methods for determining V_{(onset fraction T)} for the comingled stock tank oil are slightly more complicated, since it is not appropriate to merely use % blending of the values for the separate stock tank oils which form the comingled stock tank oil.

In some instances, V_{(onset fraction T)} for the comingled stock tank oil with each titrant may be determined from the asphaltene critical solvent power for the comingled stock tank oil with each titrant, CSP_{(blend STO+T)}, for instance according to formula 13:

V_{(onset fraction T)}=(1−(CSP_{(blend STO+T)}/SP_{blend STO})*100 (13)

The solvent power of the comingled stock tank oil, SP_{blend STO,} is calculated by volume % blending of the solvent powers of the separate stock tank oils that form the comingled stock tank oil.

Where the comingled stock tank oil is made of up of n separate stock tank oils, CSP_{(blend STO+T)} for the comingled stock tank oil with each titrant may be determined from the critical solvent power of each of the separate stock tank oils with each titrant, CSP_{(separate STO+T)}, according to formula (14):

$${CSP}_{( blend STO + T )} = {\sum }_{n = STO 1}^{n} \frac{Asp {contribution}_{( separate STO )}}{Asp {content}_{( blend STO )}} * {CSP}_{( separate STO + T )} \qquad ( 14 )$$

The asphaltene contribution from each of the separate stock tank oils, Asp contribution_{(separate STO)}, may be determined using known methods. For instance, the asphaltene content of each stock tank oil may be determined from the PVT data, or it may be determined by carrying out a crude oil assay on the stock tank oil. The asphaltene contribution may then be calculated by multiplying the asphaltene content by the weight % for each of the separate stock tank oils that fo la the comingled stock tank oil.

The asphaltene content of the comingled stock tank oil, Asp content_{(blend STO)}, may be determined by summing the asphaltene contributions from each of the separate stock tank oils that form the comingled stock tank oil.

CSP_{(separate STO+T)} for each of the separate stock tank oils may be determined according to formula (15):

CSP_{(separate STO+T)}=(100−V_{(onset fraction T)})*SP_{separate STO}/100 (15)

SP_{separate STO} is the solvent power of the separate stock tank oils, which may be determined based on the Watson K factor.

V_{(onset fraction T)} may be determined experimentally by titrating the separate stock tank oils against titrant, as previously.

### Methods for Mitigating Asphaltene Precipitation

The asphaltene precipitation envelope may be used to identify locations in a system in which asphaltenes may precipitate. Accordingly, the method of the present invention enables mitigation and/or remediation strategies to be devised for areas which are prone to asphaltene precipitation.

In some instances, the present invention provides a method for mitigating the deposition of asphaltenes in a fluid extraction process, said fluid consisting of a stock tank oil and dissolved gas, said method comprising predicting the asphaltene precipitation envelope of the fluid using the methods described herein, and modifying the fluid extraction process so that the deposition of asphaltenes is reduced.

In some instances, asphaltene deposition may be reduced in at least one of the well-bore, the manifold, flowlines/risers and topsides. Deposition may be reduced by preventing asphaltene precipitation. For instance, pressure could be applied in the extraction system so as to maintain asphaltenes in their dissolved form. Alternatively, deposition may be reduced by modifying the system so that any precipitated asphaltene does not forms deposit. Deposition may also be reduced by modifying the comingling of fluids e.g. by modifying which separate fluids are comingled, the ratios in which the separate fluids are comingled, or the location at which separate fluids are comingled.

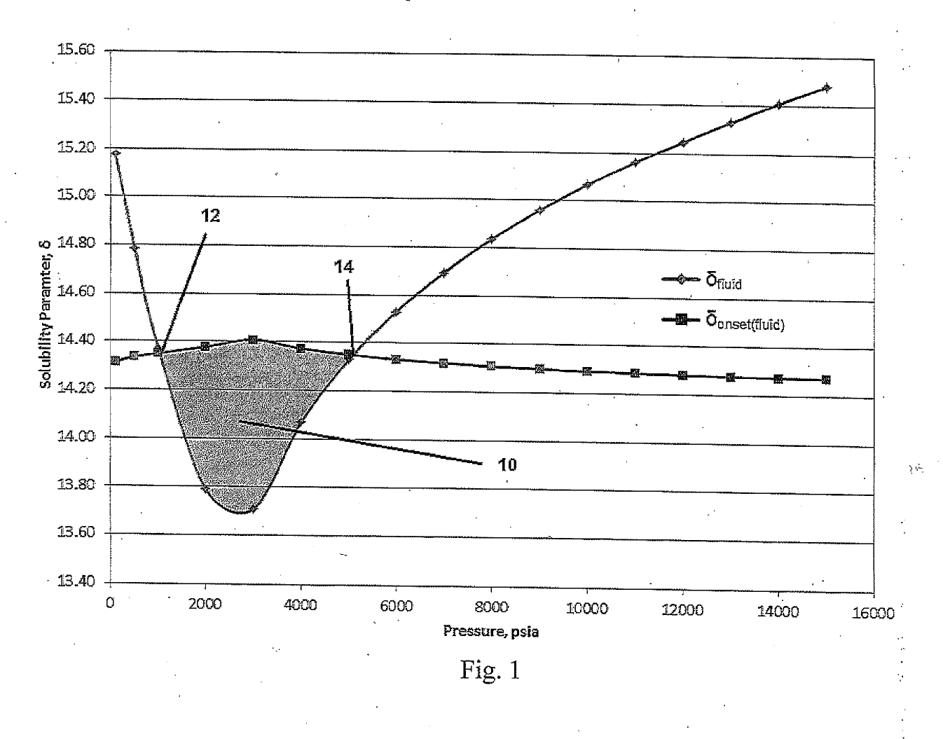

FIG. 1 depicts the asphaltene precipitation envelope (10) for a fluid. The lower asphaltene onset pressure (12) is the pressure below the oil bubble point at which asphaltenes start to precipitate from the oil. The upper asphaltene onset pressure (14) is the pressure above the oil bubble point at which asphaltenes start to precipitate. Asphaltene precipitation starts at the lower asphaltene onset pressure and occurs up to the higher asphaltene onset pressure.

It can be seen from FIG. 1 that asphaltene precipitation from the oil starts at when the pressure is reduced to around 5000 psia, i.e. the upper asphaltene onset pressure. At this point, the δ_{fluid} and δ_{onset(fluid)} profiles first intersect. Precipitation continues until the pressure reached around 1000 psia, i.e. the lower asphaltene onset pressure.

### EXAMPLES

### Example 1

### Determination of F_{correction} for a Fluid from the Gulf of Mexico (Fluid A)

Fluid A is a down-hole fluid from an oil reservoir in the Gulf of Mexico. The fluid was assessed in order to determine the correction factor, F_{correction}.

Stock tank oil was obtained from fluid A. Basic measurements were performed on the stock tank oil, as shown in Table 1:

TABLE 1
Physical properties of the stock tank oil of fluid A
Fluid A (STO)

| | |
|---|---|
| Name | Fluid A(STO) |

| | | |
|---|---|---|
| Density @ 20° C. | 0.8702 | g/cc |
| Density (Corrected to 60° F.) | 0.8733 | g/cc |
| API Gravity @ 20° C. | 31.1 | |

| | | |
|---|---|---|
| RI @ T1 | 20° C. | 1.4904 |
| RI @ T2 | 40° C. | 1.4818 |
| RI @ T3 | 50° C. | 1.4776 |
| RI @ T4 | 60° C. | 1.4733 |

An estimate of the solubility parameter based on the refractive index of the stock tank oil, δ_{STO(physical)}, was determined using formula (5):

δ_{STO(physical)}=52.042*(RI_{STO}²−1)/(RI_{STO}²+2)+2.904 (5)

At 20° C., δ_{STO(physical)}=52.042*(1.4904²−1)/(1.4904²+2)+2.904=17.96 MPa^{0.5}

An estimate of the solubility parameter based on the solvent power of the stock tank oil, δ_{STO(solvent power)}, was determined using the Watson K factor. The solvent power of the stock tank oil, SP_{STO}, was measured as 33. It is known that toluene has a solvent power of 51 and a solubility parameter of 18.2, and that heptane has a solvent power of 0 and a solubility parameter of 15.2. Using linear interpolation, δ_{STO(solvent power)} was determined at 20° C. to be 17.18 MPa^{0.5}.

The correction factor, F_{correction}, was determined according to formula (1):

F_{correction}=δ_{STO(physical)}/δ_{STO(solvent power)} (1)

### At 20° C., F_{correction}=17.96/17.18=1.045

### Example 2

### Determination of δ_{fluid} for the Fluid from the Gulf of Mexico

Fluid A was further assessed in order to determine the solubility parameter of the fluid, δ_{fluid}, across a range of pressures.

The solubility parameter of the stock tank oil, δ_{STO}, was calculated across a range of pressures from F_{correction} and δ_{STO(estimated)}, the estimate of the solubility parameter of the stock tank oil based on a physical property of the stock tank oil, according to formula (2):

δ_{STO}=δ_{STO(estimated)}/F_{correction} (2).

Since F_{correction} is independent of pressure, the value determined in Example 1 was applied across the range of pressures at which δ_{STO(estimated)} was determined.

δ_{STO(estimated)} was calculated based on the density of the stock tank oil, ρ_{STO}, according to formula (8):

δ_{STO(estimated)}=17.347*ρ_{STO}+2.904 (8)

The density of the stock tank oil, ρ_{STO}, was predicted across a range of pressures from the yield profile of the stock tank oil, as analysed using GC and high temperature simulated distillation (HT-SIMDIS), using HYSYS.

The predicted values for the density of the stock tank oil, ρ_{STO}, and the solubility parameter of the stock tank oil, δ_{STO}, are given in Table 2:

TABLE 2
Predicted values for the density of the stock tank oil, ρ_{STO},
and the solubility parameter of the stock tank oil, δ_{STO}

| Pressure, | STO Density, | |
|---|---|---|
| psia | g/cc | δ STO |
| 13000 | 0.8781 | 17.35 |
| 12500 | 0.8757 | 17.31 |
| 12000 | 0.8739 | 17.28 |
| 11500 | 0.8721 | 17.25 |
| 11000 | 0.8704 | 17.22 |
| 10500 | 0.8687 | 17.19 |
| 10000 | 0.8666 | 17.16 |
| 9500 | 0.8649 | 17.13 |
| 9000 | 0.8633 | 17.10 |
| 8500 | 0.8617 | 17.08 |
| 8000 | 0.8599 | 17.05 |
| 7500 | 0.8580 | 17.02 |
| 7000 | 0.8562 | 16.98 |
| 6500 | 0.8542 | 16.95 |
| 6000 | 0.8520 | 16.91 |
| 5500 | 0.8498 | 16.88 |
| 5000 | 0.8476 | 16.84 |
| 4500 | 0.8453 | 16.80 |
| 4000 | 0.8431 | 16.77 |
| 3500 | 0.8406 | 16.73 |
| 3000 | 0.8380 | 16.68 |
| 2500 | 0.8359 | 16.65 |
| 2000 | 0.8335 | 16.61 |
| 1500 | 0.8308 | 16.56 |
| 1000 | 0.8281 | 16.52 |

The solubility parameter of the dissolved gas, δ_{DG}, was estimated based the density of the dissolved gas, ρ_{DG}, according to formula (10):

δ_{DG}=17.347*ρ_{DG}+2.904 (10)

The density of the dissolved gas, ρ_{DG}, was determined from the composition of the dissolved gas in the live fluid, with the dissolved gas taken to be the C₁₋₆ paraffin components of the live fluid.

The composition of the dissolved gas was derived from single stage flash data in a PVT report on fluid A, and is shown in Table 3:

TABLE 3
Single stage flash data on fluid A (components are normalized
to a total of 100% for use in calculations)

| Comp | Mole % | Mol. Frac |
|---|---|---|
| N2 | 0.19 | 0.0019 |
| CO2 | 0.09 | 0.0009 |
| C1 | 76.40 | 0.7782 |
| C2 | 6.02 | 0.0613 |
| C3 | 6.47 | 0.0659 |
| i-C4 | 1.19 | 0.0121 |
| n-C4 | 3.58 | 0.0364 |
| i-C5 | 1.24 | 0.0126 |
| n-C5 | 1.62 | 0.0165 |
| C6 | 1.38 | 0.0140 |
| Totals | 98.17 | 1.00 |

Analysis of the separator flash data indicates that the C1-C6 light ends composition is very stable, except at pressures of lower than about 200 psi. The density of the dissolved gas, PDG, at different pressures was determined using Peng-Robinson equations of state based on the composition shown in Table 3.

The predicted values for the density of the dissolved gas, ρ_{DG}, and the solubility parameter of the dissolved gas, ρ_{DG}, are given in Table 4:

TABLE 4
Predicted values for the density of the dissolved gas, ρ_{DG},
and the solubility parameter of the dissolved gas, δ_{DG}

| | Gas | |
|---|---|---|
| Pressure, | Density, | |
| psia | g/cc | δ Gas |
| 13000 | 0.3811 | 9.52 |
| 12500 | 0.3771 | 9.45 |
| 12000 | 0.3729 | 9.37 |
| 11500 | 0.3685 | 9.30 |
| 11000 | 0.3638 | 9.22 |
| 10500 | 0.3589 | 9.13 |
| 10000 | 0.3537 | 9.04 |
| 9500 | 0.3481 | 8.94 |
| 9000 | 0.3422 | 8.84 |
| 8500 | 0.3358 | 8.73 |
| 8000 | 0.3288 | 8.61 |
| 7500 | 0.3213 | 8.48 |
| 7000 | 0.3131 | 8.34 |
| 6500 | 0.3040 | 8.18 |
| 6000 | 0.2939 | 8.00 |
| 5500 | 0.2826 | 7.81 |
| 5000 | 0.2696 | 7.58 |
| 4500 | 0.2547 | 7.32 |
| 4000 | 0.2373 | 7.02 |
| 3500 | 0.2166 | 6.66 |
| 3000 | 0.1919 | 6.23 |
| 2500 | 0.1627 | 5.73 |
| 2000 | 0.1294 | 5.15 |
| 1500 | 0.0942 | 4.54 |
| 1000 | 0.0597 | 3.94 |

The volume fraction of the dissolved gas, V_{(fraction DG)}, and the volume fraction of the stock tank oil, V_{(fraction STO)}, at different pressures were derived from PVT data on the live fluid, using the differential liberation residual oil density, the gas to oil ratio, the density of the stock tank oil, and the density of the dissolved gas. V_{(fraction STO)} was calculated assuming that the overall “shrinkage” of the mixture when the dissolved gas and stock tank oil are combined was fully absorbed by the gas phase.

The derived values for V_{(fraction DG)}and V_{(fraction STO)} are given in Table 5:

TABLE 5
Predicted values for the volume fraction of the dissolved gas,
V_{(fraction DG)}, and the volume fraction of the stock tank oil,
V_{(fraction STO)}

| | | Vol Frac. |
|---|---|---|
| Pressure, | Vol. Frac | Dissolved |
| psia | STO | Gas |
| 13000 | 0.6908 | 0.3092 |
| 12500 | 0.6909 | 0.3091 |
| 12000 | 0.6903 | 0.3097 |
| 11500 | 0.6896 | 0.3104 |
| 11000 | 0.6889 | 0.3111 |
| 10500 | 0.6880 | 0.3120 |
| 10000 | 0.6874 | 0.3126 |
| 9500 | 0.6862 | 0.3138 |
| 9000 | 0.6849 | 0.3151 |
| 8500 | 0.6833 | 0.3167 |
| 8000 | 0.6818 | 0.3182 |
| 7500 | 0.6802 | 0.3198 |
| 7000 | 0.6783 | 0.3217 |
| 6500 | 0.6763 | 0.3237 |
| 6000 | 0.6741 | 0.3259 |
| 5500 | 0.6716 | 0.3284 |
| 5000 | 0.6686 | 0.3314 |
| 4500 | 0.6625 | 0.3375 |
| 4000 | 0.6914 | 0.3086 |
| 3500 | 0.7217 | 0.2783 |
| 3000 | 0.7533 | 0.2467 |
| 2500 | 0.7859 | 0.2141 |
| 2000 | 0.8201 | 0.1799 |
| 1500 | 0.8560 | 0.1440 |
| 1000 | 0.8935 | 0.1065 |

Once V_{(fraction DG)}, δ_{DG}, V_{(fraction STO)} and δ_{STO} had been determined, δ_{fluid} was calculated across a range of pressures according to formula (3):

δ_{fluid}=V_{(fraction DG)}*δ_{DG}+V_{(fraction STO)}*δ_{STO} (3)

The predicted values for δ_{fluid} are given in Table 6:

TABLE 6
Predicted values for δ_{fluid}

| | | Vol Frac. | | | |
|---|---|---|---|---|---|
| Pressure, | Vol. Frac. | Dissolved | | | δ Live |
| psia | STO | Gas | δ STO | δ Gas | Fluid |
| 13000 | 0.6908 | 0.3092 | 17.35 | 9.52 | 14.93 |
| 12500 | 0.6909 | 0.3091 | 17.31 | 9.45 | 14.88 |
| 12000 | 0.6903 | 0.3097 | 17.28 | 9.37 | 14.83 |
| 11500 | 0.6896 | 0.3104 | 17.25 | 9.30 | 14.78 |
| 11000 | 0.6889 | 0.3111 | 17.22 | 9.22 | 14.73 |
| 10500 | 0.6880 | 0.3120 | 17.19 | 9.13 | 14.68 |
| 10000 | 0.6874 | 0.3126 | 17.16 | 9.04 | 14.62 |
| 9500 | 0.6862 | 0.3138 | 17.13 | 8.94 | 14.56 |
| 9000 | 0.6849 | 0.3151 | 17.10 | 8.84 | 14.50 |
| 8500 | 0.6833 | 0.3167 | 17.08 | 8.73 | 14.43 |
| 8000 | 0.6818 | 0.3182 | 17.05 | 8.61 | 14.36 |
| 7500 | 0.6802 | 0.3198 | 17.02 | 8.48 | 14.28 |
| 7000 | 0.6783 | 0.3217 | 16.98 | 8.34 | 14.20 |
| 6500 | 0.6763 | 0.3237 | 16.95 | 8.18 | 14.11 |
| 6000 | 0.6741 | 0.3259 | 16.91 | 8.00 | 14.01 |
| 5500 | 0.6716 | 0.3284 | 16.88 | 7.81 | 13.90 |
| 5000 | 0.6686 | 0.3314 | 16.84 | 7.58 | 13.77 |
| 4500 | 0.6625 | 0.3375 | 16.80 | 7.32 | 13.60 |
| 4000 | 0.6914 | 0.3086 | 16.77 | 7.02 | 13.76 |
| 3500 | 0.7217 | 0.2783 | 16.73 | 6.66 | 13.92 |
| 3000 | 0.7533 | 0.2467 | 16.68 | 6.23 | 14.11 |
| 2500 | 0.7859 | 0.2141 | 16.65 | 5.73 | 14.31 |
| 2000 | 0.8201 | 0.1799 | 16.61 | 5.15 | 14.55 |
| 1500 | 0.8560 | 0.1440 | 16.56 | 4.54 | 14.83 |
| 1000 | 0.8935 | 0.1065 | 16.52 | 3.94 | 15.18 |

### Example 3

### Determination of δ_{onset(fluid)} for the Fluid from the Gulf of Mexico

Fluid A was further assessed in order to determine the onset solubility parameter of the fluid, δ_{onset(fluid)}, at one or more pressures.

Fluid A was titrated against each of three n-paraffin titrants: heptane (C7), undecane (C11) and pentadecane (C15) at three different temperatures: 40° C., 50° C. and 60° C. The stock tank oil and the titrant were equilibrated for 30 minutes. The precipitation onset volume was determined to a precision of at least 2% by volume. The stock tank oil and titrant mixtures were observed under an optical microscope to determine when asphaltene precipitation occurs.

From the titrations, the volume fraction of the stock tank oil at the onset of asphaltene precipitation, V_{(onset fraction STO)}, the volume fraction of the titrant at the onset of asphaltene precipitation, V_{(onset fraction T)}, and the root molar volume of precipitants at the onset of asphaltene precipitation, vₚ^{0.5}_{(STO+T)}, were determined.

The solubility parameters of the titrants, δ_{T}, were deteimined experimentally based on the refractive index of each titrant at each temperature. The solubility parameter of the stock tank oil, δ_{STO}, was also determined experimentally based on the refractive index of the stock tank oil. The correction factor, F_{correction}, was applied according to formula (2) in the determination of δ_{STO}. The measured solubility parameters of the titrants, δ_{T}, and the solubility parameter of the stock tank oil, δ_{STO}, are shown in Table 7:

TABLE 7
Solubility parameters of the titrants, δ_{T}, and the solubility
parameter of the stock tank oil, δ_{STO}

| | (RI_{STO}² − 1)/(RI_{STO}² + 2) | δ STO | nC7, δ | nC11, δ | nC15, δ |
|---|---|---|---|---|---|
| 40° C. | 0.2850 | 16.96 | 14.89 | 15.75 | 16.18 |
| 50° C. | 0.2829 | 16.86 | 14.74 | 15.63 | 16.06 |
| 60° C. | 0.2807 | 16.75 | 14.59 | 15.50 | 15.94 |

Once the volume fraction of the stock tank oil at the onset of asphaltene precipitation, V_{(onset fraction STO)}, the volume fraction of the titrant at the onset of asphaltene precipitation, V_{(onset fraction T)}, the solubility parameter of the stock tank oil, δ_{STO}, and the solubility parameters of the titrants, δ_{T}, had been determined, the solubility parameter of the stock tank oil and titrant, δonset(STO+T), was determined according to formula (4):

δ_{onset(STO+T)}=V_{(onset fraction T)}*δ_{T}+V_{(onset fraction STO)}*δSTO (4)

The solubility parameter of the stock tank oil and the different titrants, δ_{onset(STO+T)}, at the different temperatures is shown in Table 8:

TABLE 8
Solubility parameter of the stock tank oil and titrant,
δ_{onset(STO+T)}, at different temperatures

| | Onset | Onset | Onset | | | |
|---|---|---|---|---|---|---|
| Temp, | C7, | C11, | C15, | δ_{onset(STO+T)}, | δ_{onset(STO+T)}, | δ_{onset(STO+T)}, |
| ° C. | vol % | vol % | vol % | C7 | C11 | C15 |
| 40 | 59.3 | 63.7 | 58.5 | 15.73 | 16.19 | 16.50 |
| 50 | 59.4 | 63.8 | 58.5 | 15.60 | 16.07 | 16.39 |
| 60 | 59.4 | 63.9 | 58.7 | 15.47 | 15.95 | 16.28 |

As shown in Table 9, the solubility parameter of the stock tank oil and the different titrants, δ_{onset(STO+T)}, and the root molar volume of precipitants at the onset of asphaltene precipitation, vₚ^{0.5}_{(STO+T)}, were determined at a reservoir temperature of 93° C. by extrapolation:

TABLE 9
Predicted values of the solubility parameter of the stock tank oil and
the different titrants, δ_{onset(STO+T)}, and the root molar
volume of precipitants at the onset of asphaltene precipitation,
vₚ^{0.5}_{(STO+T)}, at reservoir temperature

| | δ_{onset(STO+T)} | | vₚ^{1/2} | vₚ^{1/2} | δ_{onset(STO+T)} |
|---|---|---|---|---|---|
| | 60° C. | dδ/dT | 60° C. | 93° C. | 93° C. |
| C7 | 15.47 | −1.33E−02 | 12.40 | 12.75 | 15.03 |
| C11 | 15.95 | −1.20E−02 | 14.77 | 15.02 | 15.56 |
| C15 | 16.28 | −1.14E−02 | 16.86 | 17.07 | 15.90 |

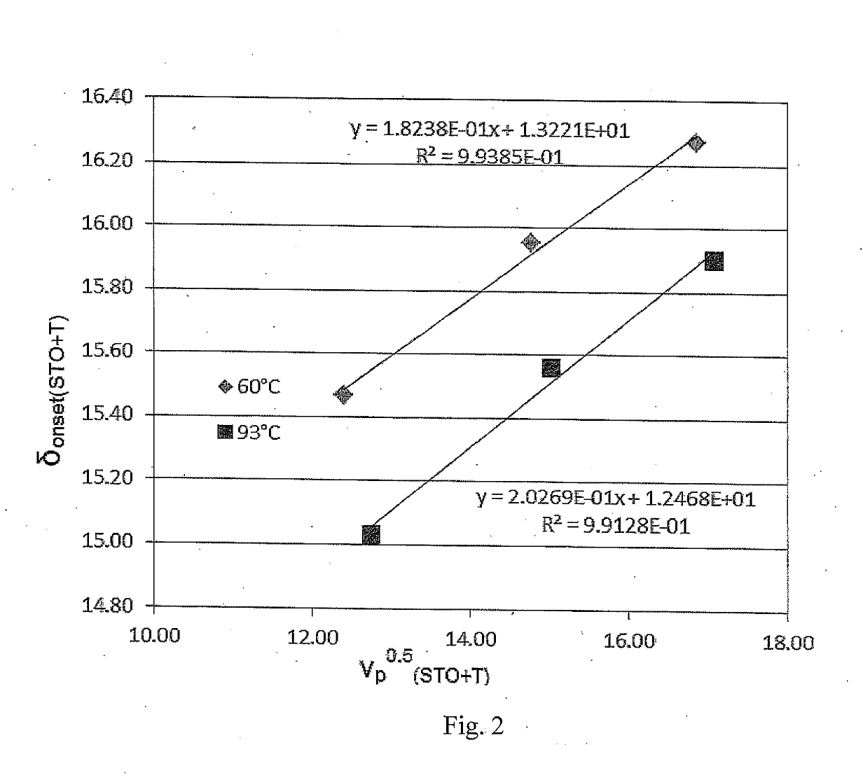

Once δ_{onset(STO+T)} and vₚ^{0.5}_{(STO+T)} at reservoir temperature had been determined for the three titrants, a relationship between δ_{onset(STO+T)} and vₚ^{0.5}_{(STO+T)} was determined. FIG. 2 shows a graph of δ_{onset(STO+T)} against vₚ^{0.5}_{(STO+T)}. It can be seen from the graph that, at reservoir temperature, the relationship between δ_{onset(STO+T)} and vₚ^{0.5}_{(STO+T)} is:

δonset(STO+T)=0.20269*vₚ^{0.5}_{(STO+T)}+12.468.

The root partial molar volume of dissolved gas in the fluid, vₚ^{0.5}_{(fluid)}, was derived from across a range of pressures from the differential liberation residual oil density and the gas to oil ratio change. The onset solubility parameter of the fluid, δ_{onset(fluid)}, was then predicted from the root partial molar volume of dissolved gas in the fluid, vₚ^{0.5}_{(fluid)}, based on the relationship between δ_{onset(STO+T)} with vₚ^{0.5}_{(STO+T)}.

The predicted onset solubility parameter of the fluid, δ_{onset(fluid)}, is shown in Table 10:

TABLE 10
Prediction of onset solubility parameter of the fluid, δ_{onset(fluid)}

| | Partial molar | | |
|---|---|---|---|
| | volume | | |
| Pressure, | dissolved gas | vₚ^{0.5} Dissolved | |
| psia | cc/mol | Gas | δ_{onset(fluid)} |
| 13000 | 59.28 | 7.70 | 14.03 |
| 12500 | 59.44 | 7.71 | 14.03 |
| 12000 | 59.72 | 7.73 | 14.03 |
| 11500 | 60.02 | 7.75 | 14.04 |
| 11000 | 60.36 | 7.77 | 14.04 |
| 10500 | 60.72 | 7.79 | 14.05 |
| 10000 | 61.06 | 7.81 | 14.05 |
| 9500 | 61.51 | 7.84 | 14.06 |
| 9000 | 62.00 | 7.87 | 14.06 |
| 8500 | 62.55 | 7.91 | 14.07 |
| 8000 | 63.12 | 7.94 | 14.08 |
| 7500 | 63.74 | 7.98 | 14.09 |
| 7000 | 64.44 | 8.03 | 14.10 |
| 6500 | 65.19 | 8.07 | 14.10 |
| 6000 | 66.01 | 8.12 | 14.12 |
| 5500 | 66.95 | 8.18 | 14.13 |
| 5000 | 68.03 | 8.25 | 14.14 |
| 4500 | 76.38 | 8.74 | 14.24 |
| 4000 | 75.24 | 8.67 | 14.23 |
| 3500 | 74.20 | 8.61 | 14.21 |
| 3000 | 73.33 | 8.56 | 14.20 |
| 2500 | 72.84 | 8.53 | 14.20 |
| 2000 | 72.70 | 8.53 | 14.20 |
| 1500 | 73.19 | 8.56 | 14.20 |
| 1000 | 75.25 | 8.67 | 14.23 |

### Example 4

### Determination of the Asphaltene Precipitation Envelope for the Fluid from the Gulf of Mexico

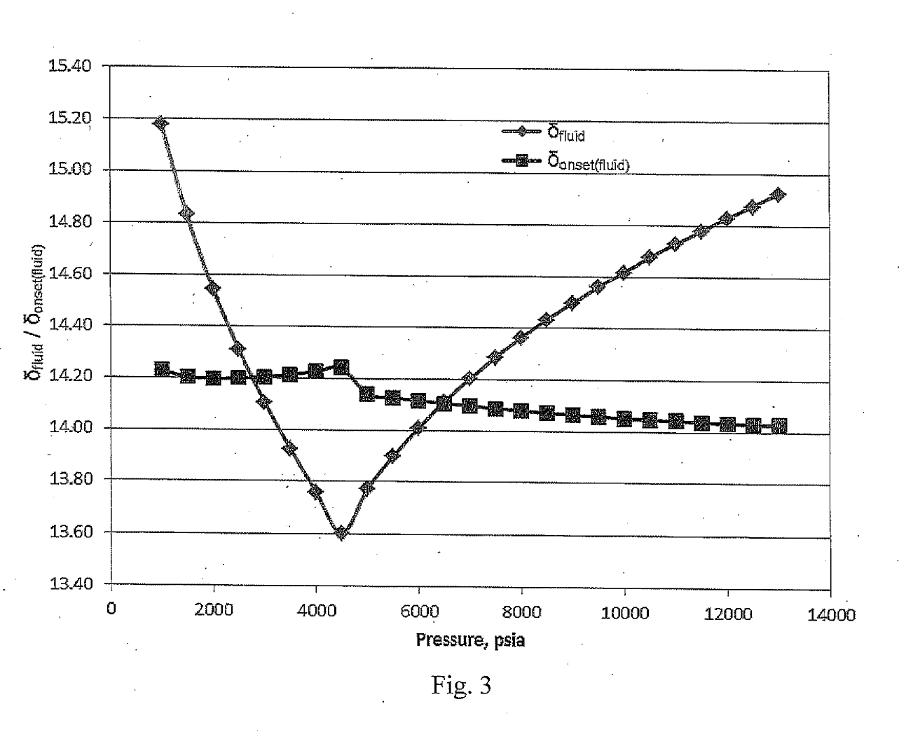

The asphaltene precipitation envelope of fluid A was predicted by comparing the solubility parameter of the fluid, δ_{fluid}, with the onset solubility parameter of the fluid, δ_{onset(fluid)}, across a range of pressures. FIG. 3 shows a graph of δ_{fluid} and δ_{onset(fluid)} across the range of pressures measured. From the graph, the upper asphaltene onset may be estimated as approximately 6,500 psi and the lower asphaltene onset pressure may be estimated as approximately 2,750 psi at a reservoir temperature of 93° C.

From SDS experimentation on a high quality live fluid sample, Fluid A is known to have an asphaltene onset pressure of 6,500 psi. The ASIST method predicted that there was no asphaltene precipitation method. Accordingly, it can be seen that the methods of the present invention may be used to predict the asphaltene precipitation from live fluids with greater accuracy than the prior art ASIST method.

### Example 5

### Other fluids

The methods outlined in Examples 1-4 were carried out on a further four fluids: fluid B, fluid C, fluid D, and fluid E. The upper asphaltene onset pressure and lower onset pressure as predicted using the method of the present invention are shown in Table 11, together with the upper asphaltene onset pressure as predicted using the ASIST method, and as measured directly from live oil (or estimated based on direct measurements on similar fluids):

TABLE 11
Upper and lower asphaltene onset pressure as predicted using the
method of the present invention, as compared to upper asphaltene
onset pressure as predicted using the ASIST method and as
measured (or estimated*)

| | | | | Measured or |
|---|---|---|---|---|
| | | | | Estimated |
| | UAOP | LAOP | ASIST UAOP | UAOP |
| Fluid A | 6,500 | 2,750 | No AOP | 6,800 |
| Fluid B | 10,000 | 8,500 | No AOP | 12,000 |
| Fluid C | 10,500 | 1,000 | 5,000 | 10,000 |
| Fluid D | 5,000 | 1,000 | 6,000 | 4,000 |
| Fluid E | No AOP | No AOP | No AOP | No AOP |

Accordingly, it can be seen that the method of the present invention provides a better estimate of the asphaltene precipitation behavior of a fluid than the prior art ASIST method.

### Example 6

### Comingled Fluids

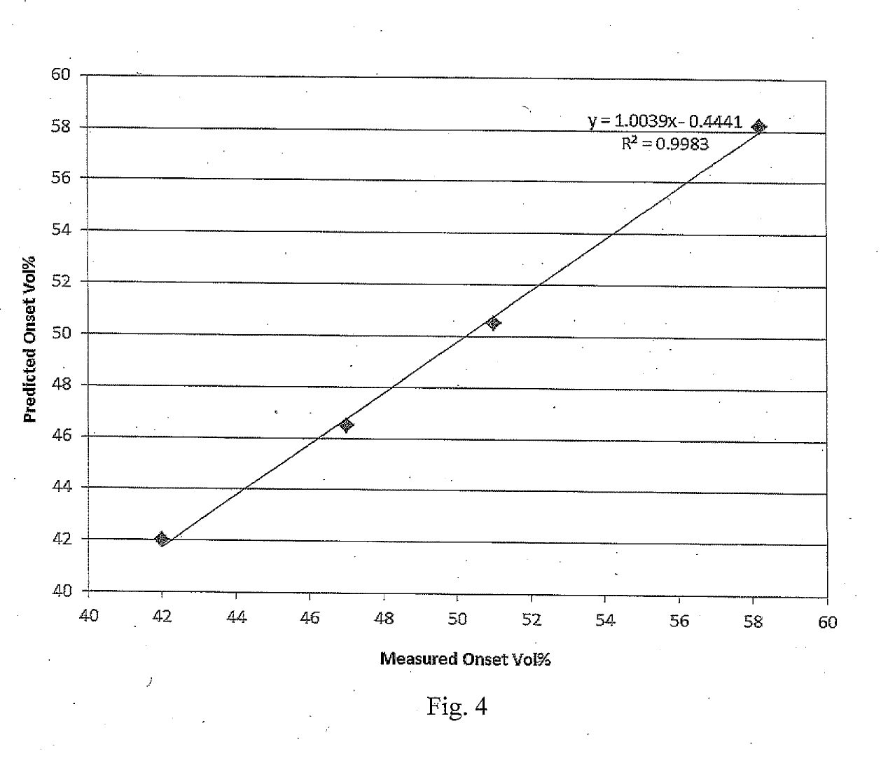

Validation of the described approach for comingled fluids was carried out by direct measurement of the onset volumes of a series of commingled stock tank oils and comparison of the predicted with the measured onset volumes with each titrant. The comparison is shown in graph form in FIG. 4 for mixtures of fluids B and C, with nC7 used as the titrant. It can be seen that there is good agreement between the predicted and measured onset volumes.

What is claimed is:

1. A method for determining the solubility parameter of a stock tank oil, δ_{STO}, at one or more pressures, said method comprising:
determining a correction factor, F_{correction}, according to formula (1):
F_{correction}=δ_{STO(physical)}/δ_{STO(solvent power)} (1)
wherein: δ_{STO(physical)} is an estimate of the solubility parameter of the stock tank oil based on a physical property of the stock tank oil, and
δ_{STO(solvent power)} is an estimate of the solubility parameter of the stock tank oil based on the solvent power of the stock tank oil,
applying the correction factor, F_{correction}, to an estimate of the solubility parameter of the stock tank oil based on a physical property of the stock tank oil, δ_{STO(estimated)}, at one or more pressures, according to formula (2):
δ_{STO}=δ_{STO(estimated)}/F_{correction} (2).

2. The method of claim 1, wherein the physical property of the stock tank oil on which δ_{STO(physical)} is based is selected from the density of the stock tank oil and the refractive index of the stock tank oil.

3. The method of claim 1, wherein the δ_{STO(solvent power)} is determined from the Watson K factor of the stock tank oil, K_{STO}.

4. The method of claim 1, wherein F_{correction} is determined at a single pressure.

5. The method of claim 1, wherein the physical property of the stock tank oil on which δ_{STO(estimated)} is based is selected from the density of the stock tank oil and the refractive index of the stock tank oil.

6. The method of claim 5, wherein the physical property of the stock tank oil on which δ_{STO(estimated)} is based is the density of the stock tank oil.

7. The method of claim 6, wherein the density of the stock tank oil at one or more pressures is predicted using the yield profile of the stock tank oil.

8. The method of claim 1, wherein the fluid is a hydrocarbon fluid which is present in a subterranean formation.

9. The method of claim 1, wherein the fluid is a comingled fluid which is formed from a plurality of separate fluids.

10. The method of claim 9, wherein the method is carried out using PVT data and stock tank samples for the separate fluids that form the comingled fluid.

11. The method of claim 9, wherein δ_{STO(physical)} and δ_{STO(solvent power)}, as used to calculate the correction factor, F_{correction}, are determined by % blending of the δ_{STO(physical)} and δ_{STO(solvent power)} values for each of the separate fluids which form the comingled fluid.

12. A method for estimating a solubility parameter of a fluid consisting of stock tank oil and dissolved gas, δ_{fluid}, at one or more pressures, said method comprising calculating δ_{fluid} according to formula (3):
δ_{fluid}=V_{(fraction DG)}*δ_{DG}+V_{(fraction STO)}*δ_{STO} (3)
wherein: V_{(fraction DG)} is a volume fraction of the dissolved gas,
δ_{DG} is a solubility parameter of the dissolved gas,
V_{(fraction STO)} is a volume fraction of the stock tank oil, and
δ_{STO} is a solubility parameter of the stock tank oil
wherein δ_{STO} is determined according to the method of claim 1.

13. The method of claim 12, wherein δ_{DG} is determined based on the density of the dissolved gas.

14. The method of claim 13, wherein the density of the dissolved gas at one or more pressures is determined from the composition of the dissolved gas.

15. The method of claim 12, wherein V_{(fraction DG)} and V_{(fraction STO)} are derived from PVT data on the fluid.

16. A method for predicting an onset solubility parameter of a fluid consisting of stock tank oil and dissolved gas, δ_{onset(fluid)}, at one or more pressures, said method comprising:
titrating the stock tank oil against two or more titrants to determine, for each titrant, a volume fraction of the stock tank oil at the onset of asphaltene precipitation, V_{(onset fraction STO)}, a volume fraction of the titrant at the onset of asphaltene precipitation, V_{(onset fraction T)}, and a root molar volume of precipitants at the onset of asphaltene precipitation, vₚ^{0.5}_{(STO+T)};
calculating a solubility parameter of the stock tank oil with each titrant, δ_{onset(STO+T)}, according to formula (4):
δ_{onset(STO+T)}=V_{(onset fraction T)}*δ_{T}+V_{(onset fraction STO)}*δSTO (4)
wherein: δ_{T} is a solubility parameter of the titrant, and
δ_{STO} is a solubility parameter of the stock tank oil;
determining a relationship between δ_{onset(STO+T)} and vₚ^{0.5}_{(STO+T)}; and
predicting δ_{onset(fluid)} from a root molar volume of dissolved gas in the fluid, vₚ^{0.5}_{(fluid)}, based on the relationship between δ_{onset(STO+T)} and vₚ^{0.5}_{(STO+T)},
wherein δ_{STO} is determined according to the method of claim 1.

17. The method of claim 16, wherein the two or more titrants are selected from the n-paraffins.

18. The method of claim 16, wherein the stock tank oil are titrant are equilibrated for a period of time of from 20-40 minutes to determine whether asphaltene precipitation has occurred.

19. The method of claim 16, wherein δ_{T} is determined based on the density or refractive index of the titrant.

20. The method of claim 16, wherein vₚ^{0.5}_{(fluid)} is derived from PVT data on the fluid.

21. The method of claim 16, wherein the method comprises titrating the stock tank oil against two or more titrants at two or more temperatures with each titrant and, by extrapolation, determining δ_{onset(STO+T)} and vₚ^{0.5}_{(STO+T)} at reservoir temperature.

22. A method for predicting an asphaltene precipitation envelope of a fluid consisting of stock tank oil and dissolved gas, said method comprising comparing a solubility parameter of the fluid, δ_{fluid}, and an onset solubility parameter of the fluid, δ_{onset(fluid)}, across a range of pressures, to predict pressures at which asphaltene precipitation will be observed, wherein a solubility parameter of the stock tank oil, δ_{STO}, is used to determine δ_{fluid} and δ_{onset(fluid)} across the range of pressures and is calculated according to the method of claim 1.

23. The method of claim 22, wherein δ_{fluid} is obtained across the range of pressures according to the method of claim 12, and δ_{onset(fluid)} is obtained across the range of pressures according to the method of claim 16.

24. A method for mitigating the deposition of asphaltenes from a fluid consisting of a stock tank oil and dissolved gas in a fluid extraction process, said method comprising predicting the asphaltene precipitation envelope of the fluid using the method of claim 22, and modifying the fluid extraction process so that the deposition of asphaltenes is reduced.
